(19) United States
(12) Patent Application Publication
(10) Pub. No.: US 2016/0364538 A1
(43) Pub. Date: Dec. 15, 2016

(54) MULTI TIME- SCALE,MULTI-PARAMETER, DIFFERENTIALLY WEIGHTED AND EXOGENOUS-OMPENSATED PHYLOLOGICMEASUREMENT ANALYSES

(71) Applicant: Dascena, Hayward, CA (US)

(72) Inventors: Ritankar Das, Hayward, CA (US); Daniel Alan Price, Seattle, WA (US); Drew Alan Birrenkott, McFarland, WI (US)

(21) Appl. No.: 14/740,259

(22) Filed: Jun. 16, 2015

Related U.S. Application Data
(63) Continuation of application No. 14/740,250, filed on Jun. 15, 2015.

(51) Int. Cl. G06F 19/00; A61B 5/00; A61B 5/0205

(52) U.S. Cl. CPC G06F 19/345; A61B 5/02055; A61B 5/7267

(57) ABSTRACT

Systems for diagnostic decision support utilizing machine learning techniques are provided. A library of physiological data from prior patients can be utilized to train a classification component. Physiological data, including time parameterized data, can be mapped into finite discrete hyperdimensional space for classification. Dimensionality and resolution may be dynamically optimized. Classification mechanisms may incorporate recognition of quantitative interpretation information and exogenous effects.

### TECHNICAL FIELD

The present disclosure relates in general to patient monitoring and diagnosis, and in particular to data processing systems for clinician diagnostic support.

### BACKGROUND

The human body maintains healthy vital physiologies through homeostasis, a complex inhibitory feedback mechanism. In the case of serious illness, a combative positive feedback cycle may exhaust the body's reserve capacity to maintain homeostasis causing homeostatic failure. Many life-threatening conditions, including heart failure, kidney failure, anaphylaxis, hemorrhaging, and hyperglycemia, result from homeostatic failure.

It is important for clinicians to accurately assess the degree of homeostatic stability of a patient in order to determine the appropriate care setting for treatment. Patient stability assessment requires the collection of a minimum dataset of information—usually qualitative observation and vital signs. The clinician then utilizes his or her expertise to make an educated decision about the stability of the patient.

In some cases, the clinician's decision is augmented with the use of an ACDS (autonomous clinical decision support) system. However, many common ACDS systems are highly inaccurate. Therefore, many unstable patients are not transferred to appropriate care until after they have experienced a life-threatening homeostatic failure. Moreover, patients are sometimes misidentified as stable and transferred to less acute care where they undergo homeostatic failure. Collectively, these mistakes are referred to as patient transfer and discharge (TD) errors. Each year, TD errors cause vast amounts of expense and large volumes of negative patient outcomes, including deaths.

In view of this, it would be desirable to better utilize available information regarding a patient in order to improve a clinical service provider's ability to evaluate a patient's stability. Additionally, available patient data streams may be useful for assisting with other activities involving evaluation of a patient's condition, such as diagnoses of other conditions and prospective care recommendations.

### SUMMARY

The present disclosure describes, amongst other things, systems, apparatuses and methods for providing diagnostic decision support, in which physiological data from prior patients is used to train a classification component. The results of this training can be used to analyze future patient physiological data towards evaluating a wide variety of patient conditions. Conditions evaluated for decision may be binary in nature (e.g. is the patient expected to be hemostatically stable or unstable, is the patient suspected to be at risk of sepsis or not?). In other embodiments, outcome classifications may be greater than binary in nature (e.g. to which of multiple hospital wards should the patient be transferred?) or even evaluated along a continuous range (e.g. how much fluid should be supplied to a particular hypotensive patient?).

In some embodiments, the classification component maps patient descriptors comprising patient physiological data, each associated with one or more known outcomes, into one or more finite discrete hyperdimensional spaces (FDHS). Supervised machine learning processes can be applied to the mapped descriptors in order to develop a classification mechanism, such as an association between location within the FDHS and patient outcome. The derived classification mechanism can then be applied within an evaluation environment to evaluate patient descriptors associated with new patients whose future outcome is yet to be determined.

In some embodiments, multiple different FDHS and associated classification mechanisms can be defined for evaluation of a single condition. The multiple outcomes can then be aggregated into a single result, such as by averaging. In some embodiments, multiple different conditions can be mapped within a single FDHS, such that during evaluation, results for each condition can be identified by referencing a current patient descriptor within a single FDHS.

In some embodiments, it may be desirable to adjust the dimensionality and granularity of the FDHS in order to, e.g., maximize the statistical disparity between position and negative outcomes for a given condition. The dimensionality and granularity of the FDHS can be adjusted dynamically, such as via a breadth-first nodal tree search.

In some embodiments, the significance to a classification mechanism of physiological data within a patient descriptor may be weighted based on the quality of the particular physiological data. For example, measurements obtained directly from patient monitoring equipment within an electronic health record may be given greater weight than clinician notes evaluated via natural language processing.

Patient descriptors may include quantitative state data and quantitative interpretation data, either or both of which may be utilized as inputs to a classification mechanism. In some circumstances, quantitative interpretation data may be input into a patient descriptor by clinicians. In some circumstances, quantitative interpretation data may be derived from quantitative state data, and a classification mechanism may act on either or both of the quantitative state data and derived quantitative interpretation data.

Patient descriptors may include time series physiological data. Patient descriptors with time series data may be mapped into a finite discrete hyperdimensional space (FDHS) as trajectories, which trajectories may be acted upon by a classification mechanism to evaluate a patient condition. In some embodiments, the FDHS may be divided into a series of regions, and a patient's physiological data may be characterized by the series of regions through which the trajectory passes. Different mechanisms may be used for dividing the FDHS into regions, including: fixed granularity in a fixed number of dimensions; or dynamic subdivision, which may be optimized for factors such as statistical significance.

Some implementations using time series data may incorporate time-based weighting of trajectories, in which, for example, more recent physiological measurements may be given greater weight in a classification mechanism than older physiological measurements.

Multi-time scale monitoring can be utilized, in which classification results may be evaluated using multiple different time scales. A time scale may be selected based on one or more criteria, including, inter alia, the extent to which it optimizes output quality (e.g. differentiation between positive and negative outcomes during application of training data) and minimizes computational load.

Some implementations may differentiate between exogenous and endogenous changes. During training of a classification mechanism, information indicative of exogenous intervention within a patient descriptor can be utilized to associate a patient trajectory with a corresponding exogenous event. Subsequent application of the classification mechanism to a new patient descriptor may enable automated identification of an exogenous shift.

Various other objects, features, aspects, and advantages of the present invention and embodiments will become more apparent from the following detailed description, along with the accompanying drawings.

### BRIEF DESCRIPTION OF THE DRAWINGS

FIG. 1A is a process flowchart for implementing a machine-learning based diagnostic support system.

FIG. 1B is a further process flowchart for implementing a machine-learning based diagnostic support system.

FIG. 1C is a further process flowchart for implementing a machine-learning based diagnostic support system.

FIG. 2 is a schematic block diagram of a diagnostic support system training mechanism implementing supervised machine learning techniques.

FIG. 3 is a process flowchart for system training.

FIG. 4 is a schematic block diagram of a clinical diagnostic support system.

FIG. 5 is a schematic block diagram of a point of care computer.

FIG. 6 is a schematic block diagram of a decision support system server.

FIG. 7 is a process flowchart for executing an evaluation of a patient descriptor.

FIG. 8 is another process flowchart for executing evaluations of patient descriptors.

FIG. 9 is a process flowchart for dynamic multiparameter calibration.

FIG. 10 is a process flowchart for a technique for modification of matching criteria in a dynamic multiparameter calibration.

FIG. 11 is a schematic block diagram of an exemplary breadth-first nodal tree search multiparameter calibration.

FIG. 12 is a process for training a mechanism that subdivides finite discrete hyperdimensional space to map patient descriptor trajectories over time.

FIG. 13 is a process for executing assessments using the mechanism of FIG. 12.

FIG. 14 is a diagram of a process for dynamic FDHS subdivision.

FIG. 15 is a schematic block diagram of a classification mechanism compensating for endogenous and exogenous effects.

FIG. 16 is a schematic block diagram of a classification mechanism implementing multi-time scale monitoring.

### DETAILED DESCRIPTION OF THE DRAWINGS

While this invention is susceptible to embodiment in many different forms, there are shown in the drawings and will be described in detail herein several specific embodiments, with the understanding that the present disclosure is to be considered as an exemplification of the principles of the invention to enable any person skilled in the art to make and use the invention, and is not intended to limit the invention to the embodiments illustrated.

Embodiments described herein may be useful in autonomously assessing the stability of a patient and utilizing this information to make appropriate care recommendations. Systems and methods acquire and utilize existing streams of data, particularly in acute care settings (whether hospital, clinical, emergency or ambulatory) in order to provide clinical decision support to clinicians. In some applications, such support systems may complement decision methodologies utilized by clinicians by rapidly assessing a large volume of data, supplementing qualitative observations by human clinicians. In some embodiments, a clinical decision support system utilizes a repository of data describing prior patients to associate sets of quantitative data with particular outcomes. Such embodiments may compare quantitative information from a patient to the set of information in the repository to generate predictions on probable outcomes.

Preferably, embodiments enable evaluation of interdependencies among patient information in order to make an estimate of a patient's outcome without intervention. Many existing systems for clinical decision support utilize information independently rather than dependently. For example, the prior art MEWS (Modified Early Warning Score) technique assigns a ‘risk’ score to each vital sign measured on the patient (the higher the risk score the more likely the patient is to suffer a serious medical complication). In MEWS, the risk score can be either 0, 1, 2 or 3 for each vital sign (of which there are 6), thus the most dangerous possible score is 18 while the safest score is 0. A patient with a highly deviatory heart rate and respiration rate and normal other vital signs will have a MEWS score of 6. A patient with a highly deviatory heart rate and blood pressure and normal other vital signs will also have a MEWS score of 6. However, it may be the case that, statistically, the condition of combined deviation in heart rate and blood pressure is much more dangerous than the condition of combined deviation in heart rate and respiration rate. In that case, these patients would preferably not be assigned the same risk score, however, there is no way of identifying/utilizing such information interdependencies with the MEWS system. Informational interdependency may be an important concept for many diagnostic applications because the human body is a collection of several organ systems that function interdependently to maintain health. While clinicians effortlessly perform an analysis of the interdependent implications of several qualitative pieces of information on the patient, some embodiments of platforms described herein perform such an analysis on quantitative pieces of information on the patient by comparing a set of combined quantitative readings on the current patient with a repository of past client data to identify patients who demonstrated similar combined quantitative readings, and then utilize their outcomes to estimate the outcome of the current patient.

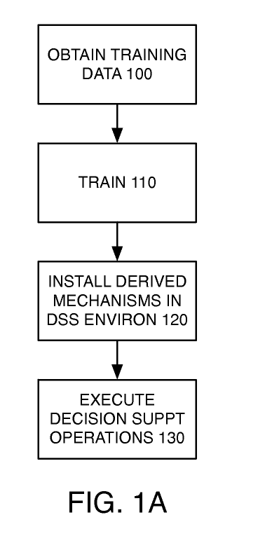

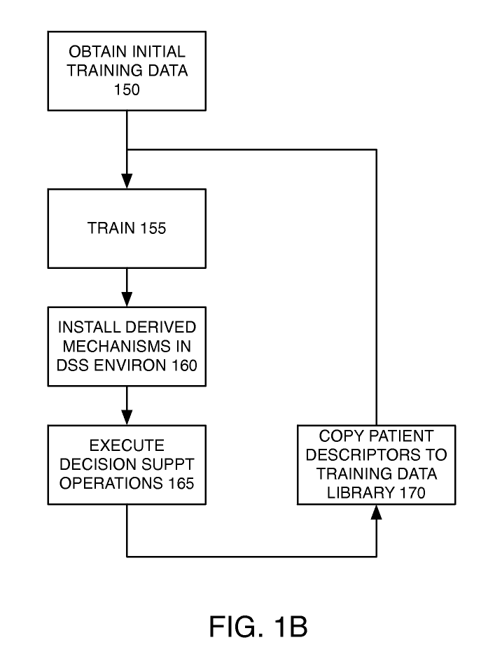

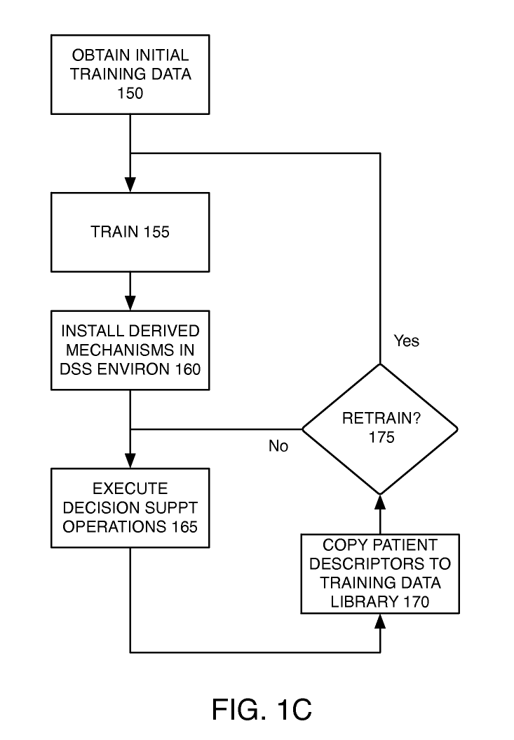

FIG. 1 illustrates, at a high level, a process that can be utilized in connection with embodiments described herein in order to implement a decision support system in a clinical environment. In step 100, a set of training data is obtained. In some embodiments, training data may be obtained as data sets published in connection with research. In some embodiments, training data may be obtained by clinical service providers in monitoring actual patients. One or more of these, or other mechanisms, may be utilized in step 100 to assemble a set of data that can be used for training. In each case, preferably the training data provides numerous patient descriptions and evaluated outcomes, with the patient descriptions each being comprised of numerous physiological measurements, potentially including time series measurements.

In step 110, supervised machine learning techniques are applied to the training data in order to derive an algorithm that is predictive of conditions or future patient outcomes, as described further below. In step 120, the algorithm derived in step 110 is installed with a diagnostic decision support system environment. In step 130, decision support operations are executed.

### System Training and Optimization

Training step 110 applies supervised machine learning methods to training data with records that include physiological data streams and actual outcomes for each of a plurality of prior patients. Supervised machine learning methods that may be employed in various embodiments of step 110 include, without limitation, linear regression, non-linear regression, Bayesian modeling, Monte Carlo methods, neural networks, random forests, k-means clustering, and process control (e.g. PID or Proportional-Integral-Derivative controller). Training step 110 seeks to identify correlations between physiological data inputs and eventual patient outcomes, preferably through application of nonlinear analysis methods known in the art, such as combinations of regression, curve fitting, fuzzy functions and pseudorandom optimizations. In some embodiments, a decision support system is trained to generate a set of algorithm calibration constants that, when applied to a patient descriptor in a clinical environment implementing the same algorithm, are predictive of one or more patient outcomes or supportive of one or more decisions. A subset of the data within the patient descriptor may be utilized for analysis, where the subset includes some or all of the data within the patient descriptor, and/or data derived from data within the patient descriptor.

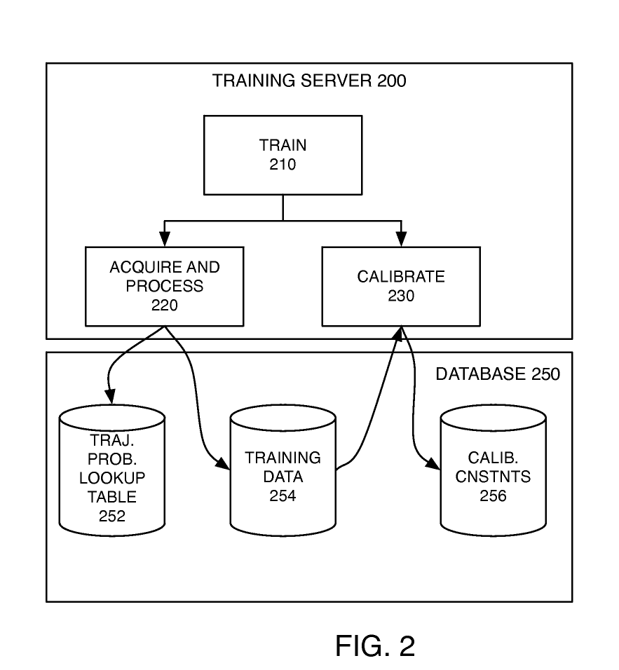

In some embodiments, steps 100 and 110 may be performed outside the clinical environment, such as by an equipment manufacturer or service provider. FIG. 2 is a schematic block representation of a computing environment that may be utilized in order to perform steps 100 and 110, according to some embodiments. Training server 200 communicates with database 250, such as via a local area computer network. Database 250 includes trajectory probability lookup table data store 252, training data store 254 and calibration constant data store 256. Training server implements train component 210, acquire and process subcomponent 220 and calibrate subcomponent 230.

While elements within the embodiment of FIG. 2, and other embodiments described herein, are illustrated in particular configurations, it is understood and contemplated that in other embodiments, functional elements may be implemented in different configurations, as would be known to a person of ordinary skill in the art of information system design. For example, elements may be implemented using different hardware or software abstractions, such as distributed hardware or software resources, virtual machines, containerized applications, and other configurations. The servers and other computing devices may include a variety of physical, functional and/or logical components. That said, the implementation of the servers and other computing devices will preferably include, at some level, one or more physical computers having one or more microprocessors and digital memory for, inter alia, storing instructions which, when executed by the processor, cause the computer to perform methods and operations described herein.

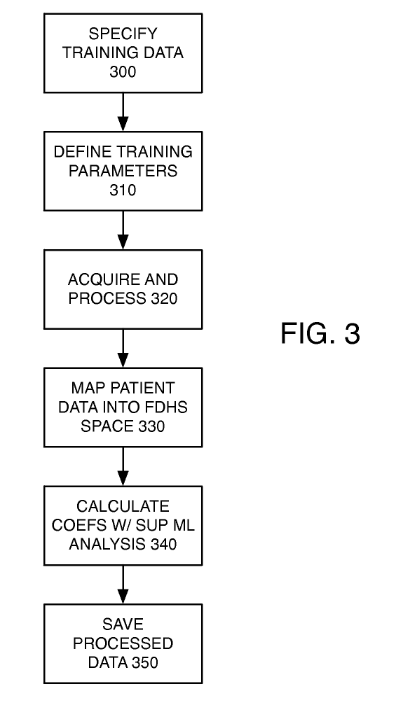

FIG. 3 illustrates an embodiment of a process for implementing step 110 (training a decision support algorithm) within the environment of FIG. 2. In step 300, a system operator specifies a target path for a training dataset within training data store 254, and a data subset to be utilized for processing (if any), via interaction with acquire and process subcomponent 220. Typically, training data will include, for each of a plurality of prior patients that have been observed, patient descriptors that include physiological information, along with a prior outcome determination.

In step 310, training parameters are defined. Initial training parameters may include trajectory method parameters (described further below). Initial training parameters defined in step 310 may also include calibration parameters, such as number of unique calibration constants to analyze, starting points for calibration constants, and a number of recursive analysis layers to perform. In step 320, acquire and process component 220 accesses the data set specified in steps 300 and 310, and performs any desired data conversion and feature extraction. In step 330, patient data from the training set is mapped into Finite Discrete Hyperdimensional Space (FDHS) by train component 210 within training server 200. In step 340, a supervised machine learning algorithm implemented by train component 210 is executed on the mapped data in order to calculate coefficients for a classification algorithm that is predictive of the desired outcome criteria based on patient descriptor input. In some embodiments, different locations within the FDHS are associated with different probabilities of a patient having a condition.

The process of FIG. 3 can be utilized to generate an algorithm capable of making any of a variety of condition determinations including, without limitation: a prediction of whether a patient is expected to experience a homeostatically stable or unstable outcome; whether a patient is likely to experience sepsis; whether a patient is likely to experience acute coronary syndrome; the amount of fluids that should be administered to maximize likelihood of homeostatic stability; which of multiple hospital wards would be best for patient transfer; and other determinations. In many such embodiments, it may be desirable to utilize time series data, such that the algorithm can analyze the progression of various physiological attributes over time towards determining a predicted future outcome. In such embodiments, the supervised machine learning algorithm in step 340 can then generate a trajectory probability lookup table within data store 252. The trajectory probability lookup table is a data structure that associates particular trajectories with a probability of either a stable or unstable outcome. Step 340 may also generate a calibration constant data structure within calibration constant data store 256.

In some embodiments of step 330, the patient data from the training set may be mapped into multiple different FDHS. Then in step 340, the supervised machine learning component may be trained within each of the FDHS to predict the desired condition. A result compilation component within train component 210 aggregates results from each FDHS to generate an outcome. Results from different FDHS may be aggregated using a variety of methods, such as averaging or weighted averaging.

In step 350, the processed training data and outcomes are stored into database 250. In some embodiments, it may be desirable to prune data that is of low significance. More specifically, some patient data trajectories may be within the training dataset which have not been observed a sufficient number of times to have statistically significant outcome associations. In such cases, it may be desirable to prune those trajectories of low significance, such as by removing them from trajectory probability lookup table 252.

The systems, methods and frameworks described herein are broadly applicable to effective implementation of a wide variety of risk assessment and decision support systems. Depending on the particular analysis being performed, certain analysis methods may be beneficially employed in maximizing the effectiveness of the resulting algorithm.

In some embodiments, it may be desirable to train classification components within multiple different FDHS for a single condition.

In some embodiments, curve fitting techniques may be effectively utilized, incorporating both linear and nonlinear components. For example, a particular physiological data dimension that is initially measured on a continuous or nearly continuous scale (e.g. heart rate) may be granularized, i.e. lumped into a predetermined number of bins such as in 30 bpm increments, with training data outcomes averaged within each bin. Each bin may therefore have some correlation with an output, such as a probability of developing a condition. In some cases, it may be beneficial to simply perform a lookup of an output value corresponding to the bin in which the actual patient's corresponding data point falls. In other cases, such as where trends are present across a range corresponding to a given bin, it may be desirable to perform a curve fitting function during system training, such as a linear or polynomial fit. In that case, a patient's actual data point can be applied against the fitted curve to identify a correlated output value. The performance of each analysis technique can be evaluated to identify the optimal analysis for any given evaluation.

In some embodiments, it may be desirable to perform input parameter weighting based, at least in part, on the quality or reliability of the input data. For example, in some embodiments, a vital sign verified manually by a clinician may be upweighted, while the same vital sign taken from the bedside patient monitor may be downweighted as less reliable. In some embodiments, features extracted from measured monitor data (such as hypertension based on extended time series of elevated blood pressure) may be upweighted as derived from an initial measure, while identification of the term “hypertension” from the application of natural language processing to unstructured text may be downweighted. In some embodiments, application of such data quality-based weighting may improve the reliability of an assessment.

### Differentiation Between Quantitative State and Quantitative Interpretation

Another factor that may be important in optimizing evaluation of patient data, both during the training stage and active evaluation stage, is distinction between quantitative state parameters and quantitative interpretation. Quantitative state information may have different meaning for different patients. For example, in some embodiments, a patient's absolute systolic blood pressure reading may be utilized as a quantitative state input. However, a systolic blood pressure of 120 mmHg may be considered safe for a typical patient, but indicative of severe and dangerous hypotension in a patient who is otherwise chronically hypertensive. Therefore, in some embodiments, it may be beneficial to utilize either or both of quantitative state information (e.g. blood pressure=120 mmHg) and quantitative interpretive information (e.g. the patient exhibits chronic hypertension) as inputs to an evaluative process, so that in some analyses the quantitative interpretive information can be utilized to guide how the quantitative state information is to be interpreted.

In some embodiments, quantitative interpretive information is separated from, and in some circumstances derived from, quantitative state information. During the training process, quantitative interpretive stored within the training data set may be utilized as a separate feature in the feature extraction step, which feature may be applied as one of the inputs to the machine learning algorithm. For example, the training data set may contain information indicative of whether each patient has, within their medical history, a prior diagnosis of hypertension. This data, which may be a binary yes/no value, may be utilized as a feature.

In some embodiments, quantitative interpretive information may be extracted computationally from quantitative state information, particularly given the availability of time series data within the patient descriptor. For example, for a feature indicative of whether a patient suffers from hypertension, server 400 may algorithmically evaluate blood pressure readings for a patient over time; if an average blood pressure over time exceeds a threshold level and the standard deviation in blood pressure readings falls below a predetermined level (indicating that blood pressure is elevated and stable), a feature may be extracted that is a positive indicator for likely hypertension. This derivation of quantitative interpretive information from quantitative state information can be applied to patient descriptors within the training data library, and/or to a patient descriptor for the patient under evaluation. In other embodiments, it may be desirable to pre-process high-frequency metrics into higher level descriptive metrics that can be fed into a classification algorithm, in addition to or in lieu of normalizing time scales between data streams or other time-based multiparameter analysis techniques described elsewhere herein. Such pre-processing may be effective in reducing the dimensionality and computational load of an evaluation compared to directly processing high-frequency time series data; it may also lead to greater correlation between classifier output and observed results.

### Application to Non-Binary Decision-Making Processes

In some embodiments, the output of training server 200 can be configured to be predictive in binary or non-binary decision-making processes. While many decisions are commonly thought of as binary decisions (e.g. is the patient stable or not?), many real world decision-making efforts are in fact of higher order: ternary, quaternary or even continuous values on a spectrum. Examples of a non-binary decision-making process that may be made in the hospital are “which ward to transfer a patient after being discharged from the emergency room?”, or “what quantity of fluid should be administered to a hypotensive patient in need of fluid resuscitation?”.

To accommodate the non-binary nature of the real world, some embodiments may utilize an analysis methodology augmented to perform N-outcomes decision-making in order to best fit the medical context. The training step 110 is comparable to the binary outcome case, except that the outcome value for each particular patient (e.g. the output of step 340 in the embodiment of FIG. 3) can range along a granular dimension (e.g. whole numbers from 0 to N) rather than only taking on binary values (e.g. 0 or 1). The lookup tables that are constructed in step 350 after the training process contain N probabilities for each trajectory—the probability of the particular trajectory yielding each of the N possible outcomes.

In the execution stage (step 130), the patient being tested is assigned an outcome score which is then mapped to one of the N outcomes. For embodiments in which the N-outcome measurement can be ordered along a continuum (e.g. the amount of fluid that should be administered to a hypotensive patient, or the number of hours in which the patient is most likely to experience ACS), the patient's outcome score exists in a 1-dimensional space, and the choice of the N outcomes to be assigned to the patient is determined by identifying which of the N outcomes is closest to the patient's outcome score. In the non-continuum case (e.g. which of N wards should the patient be transferred to?), the patient's outcome score exists in an N-dimensional space. However, again the choice of the N outcomes to be assigned to the patient is determined by identifying which of the N outcomes is the closest (as defined by a degree-N vector) to the patient's outcome score.

### Implementation in the Clinical Environment

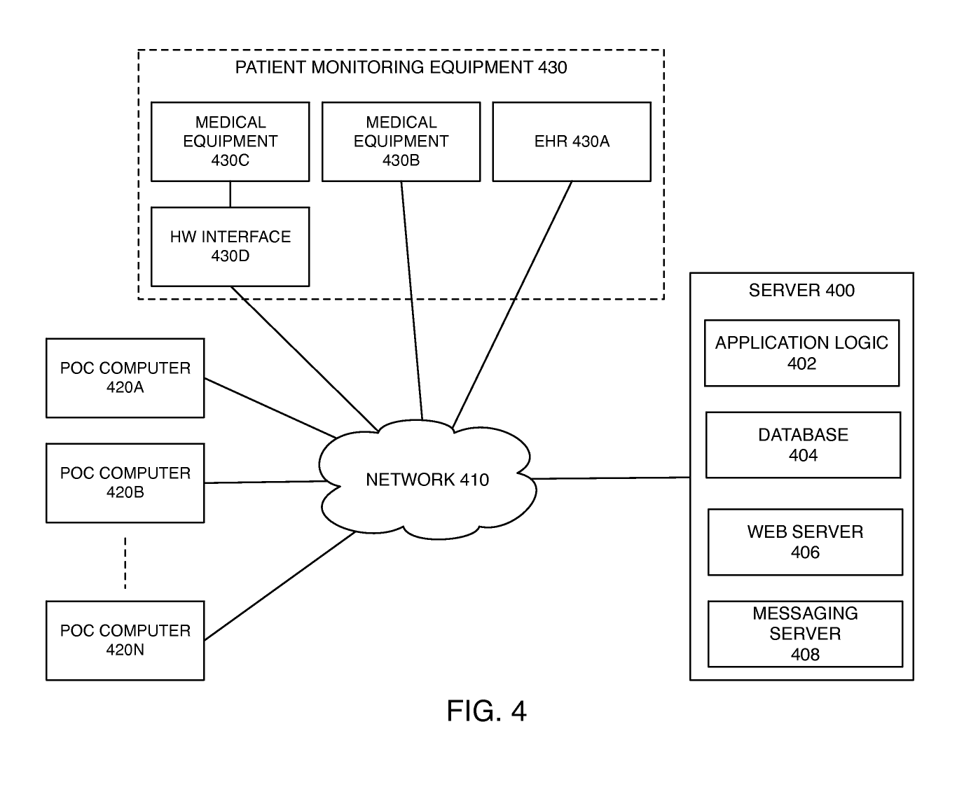

The patient outcome prediction and decision support mechanisms derived in steps 100 and 110, described above, can subsequently be implemented within a clinical environment (step 120). FIG. 4 is a schematic representation of an illustrative computing environment in which some embodiments of a clinical decision support platform may be implemented. Server 400 communicates with one or more point of care (POC) computers 420 via network 410. In some embodiments, POC computers 420 will each be installed at the point of patient care, such as a hospital room or on a movable bed. In some embodiments, POC computer 420 will be centrally located and utilized for multiple patients, such as a central ward monitoring computer installed for use with multiple patients in a particular hospital ward. In other embodiments, POC computer 420 will be a mobile device, such as a tablet computer utilized by health care service providers while moving within a facility. In some embodiments, such as for home monitoring or other monitoring of a mobile patient, POC computer 420 may be remotely located from the patient. In environments such as continuous monitoring services, POC computer 420 may even be remotely located in a different country from the patient. POC computer 420 could be installed within an ambulance, or in a triage facility for incoming ambulatory patients.

Network 410 typically includes a medical service provider facility data network, such as a local area Ethernet network. In order to maximize data security and minimize opportunities for outages in communications, in many embodiments server 400 will be installed within a medical service provider's facility, such as a hospital data center, which will be connected with each POC computers 420 within the medical care facility. However, it is understood that in other embodiments, it may be desirable to install server 400 even more remotely from POC computers 420. For example, server 400 could be installed within an off-site data collocation center, while POC computers 420 may be located within a medical care facility. In other embodiments, server 400 may be installed within a hospital headquarters facility, while POC computers 420 may include computers located at remote care sites, such as local clinics or branch facilities. In such embodiments, network 410 may include various combinations of the Internet, a private WAN, VPNs and other preferably secure data connections.

Server 400 and/or POC computers 420 communicate with one or more pieces of patient monitoring equipment 430. Patient monitoring equipment 430 may include multiple pieces of electronic equipment (430A, 430B et seq.), operating to monitor, report or evaluate one or more types of physiological patient information. The mechanism by which information is conveyed from patient monitoring equipment 430 to server 400 and/or POC computer 420 may vary based on the particular piece of equipment being utilized. In many embodiments, the health care provider facility will utilize an Electronic Health Record (EHR) system 430A. EHRs are typically centralized, network-connected systems that aggregate information associated with numerous patients within a facility. In such embodiments, server 400 may query EHR 430A via network 410 in order to obtain patient descriptors for evaluation. Some patient monitoring equipment 430B may be network-connected to provide patient information independently of an EHR, in which case server 400 may also query medical monitoring equipment 430B via network 410.

Yet other equipment, particularly older equipment, may not include native software interfaces for extraction of data. Some equipment may not include any convenient data connection at all, in which case nurses, doctors or other health care providers may observe information from the monitoring equipment 430 and manually enter corresponding data into one of POC computers 420, e.g. using a keyboard, monitor and mouse. In other such circumstances, hardware interface 430D may be provided in order to extract information from medical equipment 430C and convey it in a format accessible to server 400. In some situations, hardware interface 430D may make patient information available for query via network 410. In other circumstances, hardware interface 430D may provide a local wired connection, such as a serial connection, to one of POC computers 420, which in turn reports collected information back to server 400.

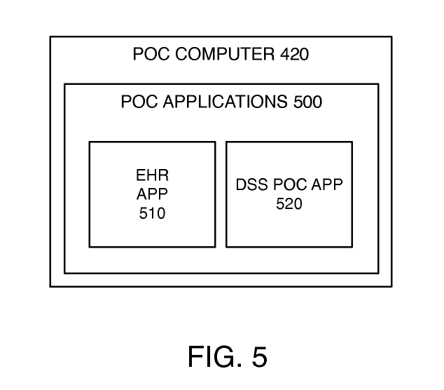

FIG. 5 illustrates an embodiment of a POC computer 420. POC applications 500 are executed on POC computer 420. POC applications 500 include EHR application 510, which enables user interaction with EHR system 430A, as well as decision support system point of care (DSS POC) application 520. In some embodiments, DSS POC application 520 is implemented as a web application, operating in conjunction with server 400. In some embodiments, EHR application 510 includes capabilities for web service integrations, such that DSS POC application 520 can operate within EHR application 510. Applications 510 and 520 enable interaction with a user of POC computer 420, such as by displaying information on a computer monitor and accepting user input via a keyboard, mouse, touchpad, touchscreen, trackball, microphone with voice-to-text conversion functionality, or other means of user interaction. In some embodiments, it may be desirable to enable users to provide input to DSS POC application 520 via other devices, such as a smartphone or tablet computer, in which DSS POC application 520 may include external device interface capabilities as are known in the art, whether via Ethernet, Bluetooth, or other digital communications link. In some embodiments, DSS POC application 520 can be implemented directly on a mobile phone or tablet computer.

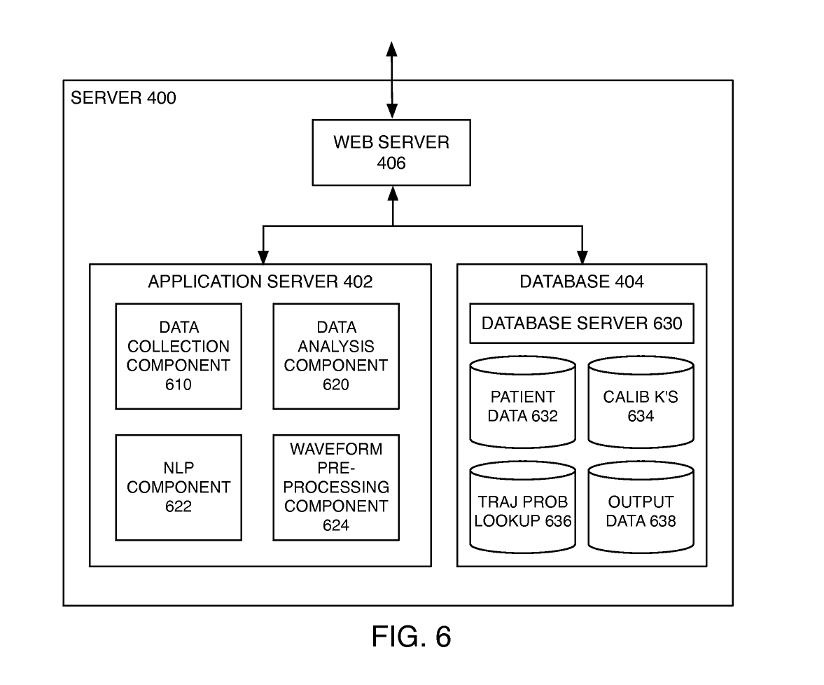

FIG. 6 further illustrates components of server 400. Web server 406 enables communications with external devices via network 410, such as POC computers 420 and patient monitoring equipment 430. Web server 406 may include, inter alia, an Application Programming Interface (API) through which data may be securely exchanged with external devices. Web server 406 may also include a web site server implementing a user interface (e.g. an HTTP-based user interface) for implementation of DSS POC application 520. Application server 402 implements software components including data collection component 610, data analysis component 620, natural language processing component 622 and waveform pre-processing component 624. Database 404 provides mechanisms for data storage, and includes database server 630, patient descriptor data store 632, calibration constants data store 634, trajectory probability lookup table 636, and output data store 638.

Prior to system use, server 400 is loaded with data describing previously-derived prediction and/or decision support mechanisms. Preferably, data analysis component 620 utilizes the same evaluation algorithm implemented during a training operation described above in connection with FIGS. 1-3. In such embodiments, calibration constants data store 634 can be loaded with the same optimized calibration constants derived by training server 200 and stored within database 256; and trajectory probability lookup table 636 can be loaded with the same optimized lookup table data derived by training server 200 and stored within lookup table 252.

While calibration constants and lookup tables are initially loaded prior to system use, server 400 is also readily upgradable to add or improve capabilities and performance. Further training iterations can be run, particularly as additional training data becomes available for analysis, even after installation and use of server 400 in an active clinical environment. Preferably, training server 200 and DSS server 400 utilize common, versatile machine learning algorithms applicable to numerous different evaluation scenarios, in which case only the contents of calibration constants data store 634 and lookup table 252 need be updated to add new analyses or upgrade the performance of existing analyses. Such updates may be done in an automated fashion, or by a system administrator.

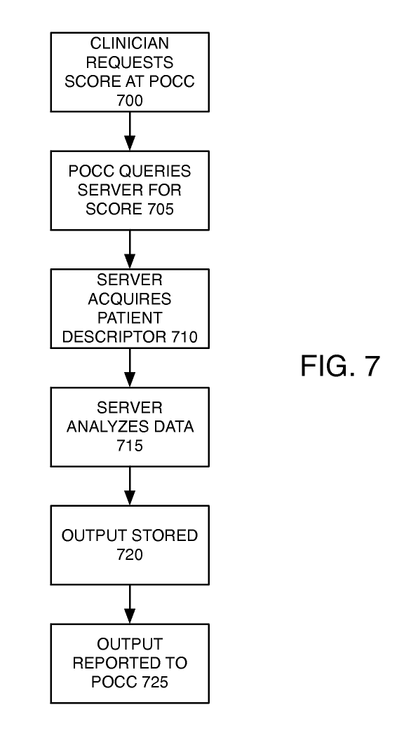

Once server 400 is configured, clinicians can utilize it to perform various assessments and evaluations. FIG. 7 illustrates one embodiment of a process by which assessments can be performed. In step 700, a clinician requests a particular evaluation using POC computer 420 and DSS POC app 520. In step 705, DSS POC app 520 queries server 400 for a result, transmitting, inter alia, a patient identifier and the nature of the analysis requested. In step 710, server 400 queries patient monitor equipment 130 for patient descriptor data corresponding to the patient identified in step 705. In step 715, server 400 analyzes the patient descriptor obtained in step 710 by applying the algorithm, calibration constants and, as appropriate, lookup table (as described above) corresponding to the particular analysis requested in step 705. In step 720, an output is generated and stored within output database 638. In step 725, the output result is returned to DSS POC application 520 and displayed for the clinician.

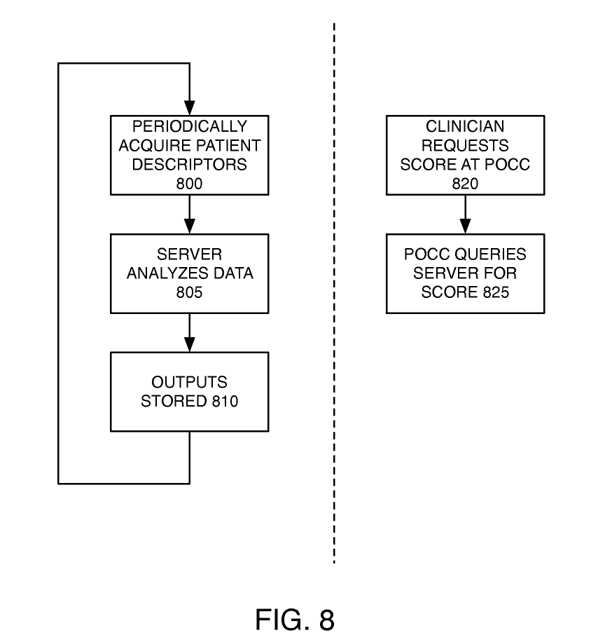

The process of FIG. 7 provides on-demand analysis, while minimizing the quantity of patient data imported into the decision support system. On the other hand, the process of FIG. 7 only calculates scores after being specifically triggered by a clinician, thereby incurring some (typically minor) delay for data aggregation and processing. Also, because scores are only calculated at discrete, manually-triggered events, such implementations may not facilitate applications in which a regular time series of scores or assessments is desired, or in which ongoing automated monitoring of a particular condition or risk is desired. To that end, FIG. 8 illustrates an alternative analysis technique in which evaluations are performed automatically on a periodic basis, with output being stored in an output database for rapid response to clinician queries and/or automated monitoring. In step 800, server 400 periodically queries patient monitoring equipment for patient descriptors for multiple patients. In step 805, server 400 evaluates each patient descriptor for one or more assessments. In step 810, the assessment outputs are stored within output database 638. The process of steps 800, 805 and 810 repeat periodically. In parallel, clinicians may query the system on demand to view assessment output. In step 820, a clinician requests a score or other evaluation output via interaction with a POC computer 420 and DSS POC application 520. In step 825, POC computer 825 queries server 400, and results database 638, for the requested patient evaluation output. Similarly, automated monitoring processes can be configured to periodically evaluate results to monitor for various conditions, such as rapid increase in likelihood of homeostatic instability, sepsis or ACS.

In some embodiments, it may be beneficial to implement both continuous evaluation processes, such as that of FIG. 8, as well as on-demand evaluation processes, such as that of FIG. 7. For example, in such an implementation, server 400 may operate to regularly, periodically perform an evaluation of the risk of hemostatic instability for each patient in a critical care ward. Meanwhile, clinicians may utilize the process of FIG. 7 to evaluate risk of sepsis for a particular patient upon request.

### Dynamic Multiparameter Calibration

In accordance with another aspect of some embodiments described herein, it may be desirable to calibrate training data to balance the quantity of patient data analyzed against the confidence of prediction outcomes. With each additional piece of information added to the coupled quantitative patient description, there will be fewer patients in any given dataset that exhibit exactly the same set of quantitative descriptors. Thus, with increasing detail on the quantitative description of the patient, there is decreasing statistical significance of the outcome prediction. Taken to the logical extreme, if a patient is described with every piece of quantitative information available at a particular point in time, there will likely be no patients in the repository that exactly match. Therefore, in some embodiments it may be important to provide a framework to optimize the tradeoff between incorporated patient information and statistical significance of the outcome estimate. At a high level, this optimization can be performed on a case-by-case basis by modifying the granularity of the quantitative measurements as well as modifying the subset of all available measurements utilized as a patient descriptor, to arrive at an optimal balance of descriptiveness and prediction statistical significance given the available patient information and available data repository.

Typical existing methodologies for medical risk stratification and patient assessment rely on a predefined set of measurement values coupled together in predefined combinations. In these methods, the dimensionality of the assessment and the granularity of the measurement axes are hardcoded into an algorithm and will be fixed constants for every patient. Such techniques present several potential drawbacks. First, patients will be irregularly distributed in the multidimensional space, e.g. more patients will exhibit groupings of biologic measurements that are closer to population averages, and fewer patients will exhibit more highly abnormal groupings of biologic measurements. Therefore, the statistical significance of a particular sample point in the multidimensional space is variable and highly location-dependent. Second, it may be unclear, prior to supervised machine learning trials, which groupings of biologic measurements are more tightly correlated with a particular patient outcome than others.

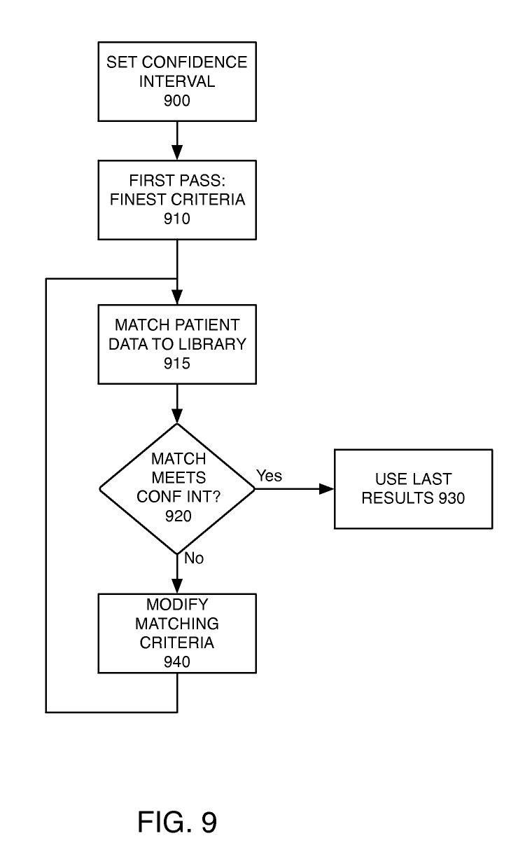

In accordance with some embodiments, a system and method may be implemented to dynamically adjust the dimensionality of each assessment, and the granularity of each measurement, for a particular patient or evaluation condition, substantially in real time. FIG. 9 illustrates such a dynamic multiparameter calibration process, which may be implemented in the systems described elsewhere herein. In step 900, a confidence interval is defined. In some embodiments, a confidence interval may be defined as the minimum number of records in the data library that must be matched to make a prediction on a patient's outcome. For example, in some types of analysis, it may be desirable to require that at least 30 records match the current patient's descriptor in order to provide a sufficient statistical basis for estimating an outcome.

In step 910, initial matching criteria are set with a finest level of granularity and highest desired number of dimensions. In step 915, the current patient descriptor is matched against the library data using the configured granularity and dimensionality. In step 920, a determination is made as to whether the number of library records matching the current patient's descriptor exceeds the threshold confidence interval set in step 900. If so, in step 930, the most recently matched records are utilized to determine the desired results (e.g. estimate the outcome of the patient). If not, the matching criteria are modified to reduce the dimensionality of the patient descriptor and/or increase the granularity (step 940). The operation then returns to step 915, and the matching operation iterates with increasingly reduced dimensionality or increased granularity until the matching results satisfy the desired confidence interval.

The matching criteria modification of step 940 can be implemented in a number of different ways. In some embodiments, the criteria can be modified randomly, such as by randomly selecting a data dimension for elimination or modification of measurement granularity. In some embodiments, the criteria can be modified quasi-intelligently, using an intelligent algorithm with a stochastic process built in. In other embodiments, the criteria can be modified intelligently, without the use of stochastic process.

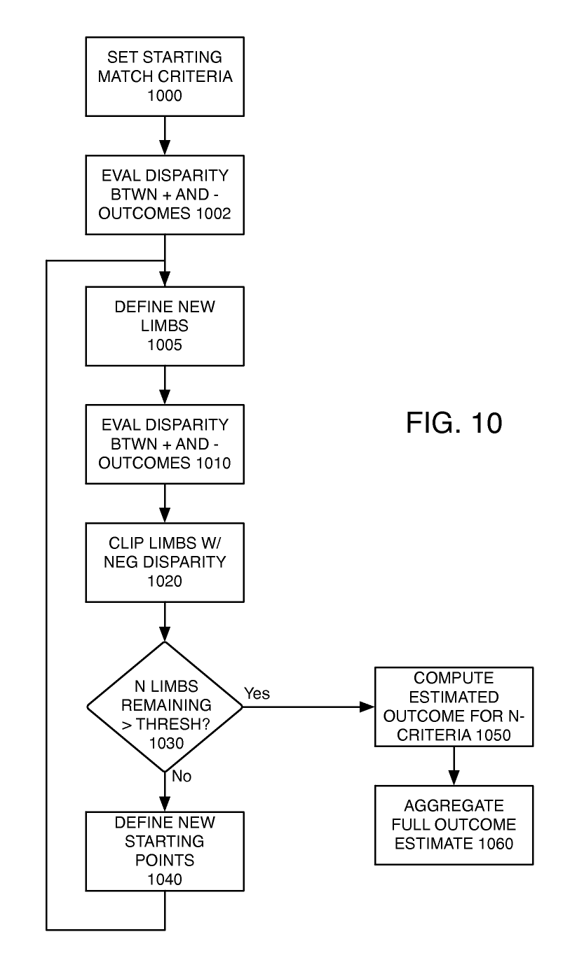

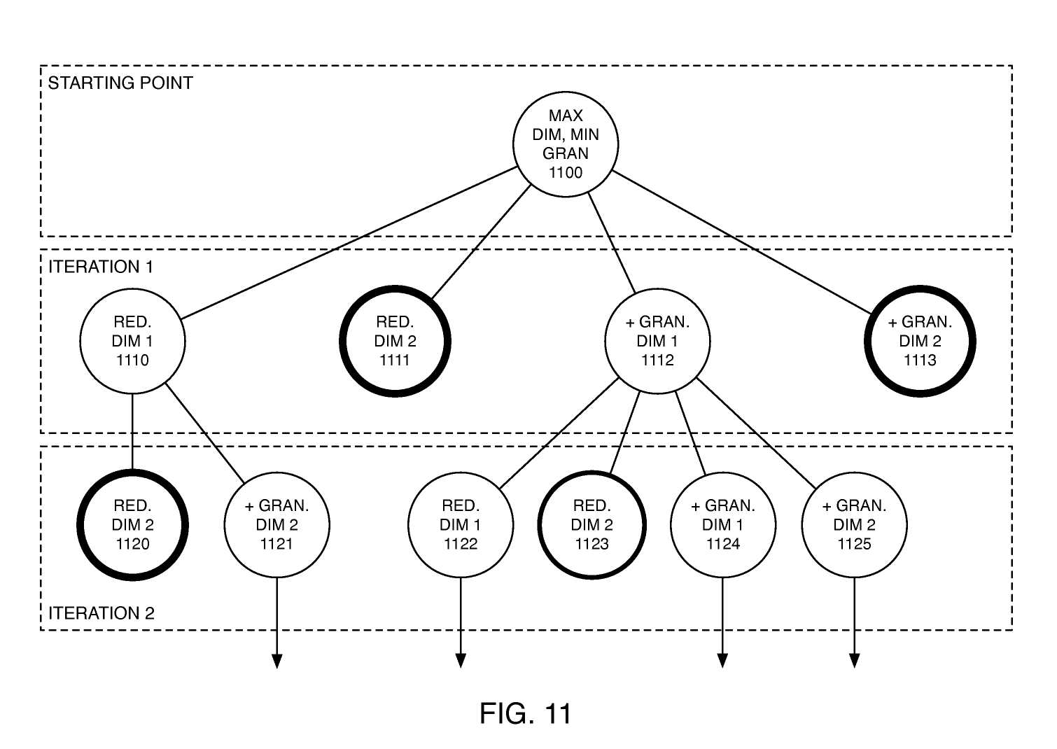

An intelligent mechanism for modification of matching criteria is illustrated in the flow chart of FIG. 10 and nodal tree of FIG. 11. The technique of FIGS. 10 and 11 is a breadth-first nodal tree search of the various singular modifications of the matching criteria that are available (i.e. drop one dimension, or change the granularity of one dimension). In step 1000, a matching starting point is determined as the finest level of granularity and maximum number of dimensions. In step 1002, an evaluation is performed of the disparity between the clustering pattern of records with negative outcome from patterns of records with a positive outcome (described further below).

In step 1005, new nodal tree limbs are defined with each of multiple options for reducing dimensionality or increasing granularity relative to the most recent starting point. The mechanism seeks to identify N limbs with a threshold significance, e.g. a minimum number of prior patient descriptors corresponding to the node. In step 1010, for each new limb, an evaluation is performed of the disparity between the clustering pattern of records with negative outcome from patterns of records with a positive outcome. Examples of an increase in the disparity of clustering patterns of records with a negative outcome from patterns of records with a positive outcome include: an increase in the distance between the means of the two datasets; a difference in the standard deviation of the two datasets; and an observable pattern emergent in the logistic regression comparison of the two datasets.

Limbs in which the disparity decreases will be clipped (step 1020), as the modification of dimensionality and/or granularity appears to have decreased the statistical confidence level of the result. Limbs in which the disparity increases are considered positively, as the apparent statistical correlation with outcome is increased. In step 1030, the N remaining nodes having greatest disparity are checked to see if they meet the threshold significance. If not, the N nodes with greatest disparity become the bases on which to perform further breadth-first searching in a further iteration (step 1040). The process returns to step 1005 in order to define new limbs, each having a further reduction in dimensionality or increase in granularity relative to the remaining limbs in the prior iteration.

This search can be continued until there are at least N sets of matching criteria that all meet the confidence interval defined in step 1000. At that point, in step 1050, estimated outcomes are computed based on each of the N sets of matching criteria. In step 1060, an overall estimated outcome is determined by a results compilation component within data analysis component 620 and server 400. The results compilation component may be utilized in embodiments in which a final outcome is compiled based upon multiple individual results via different analysis methods. Various techniques for compiling multiple individual results can be implemented, such as averaging the multiple results, or averaging the results after removing high and low outliers. In the embodiment of FIG. 10, the results compilation component aggregates the multiple results calculations in step 1050.

FIG. 11 provides a schematic illustration of an example of the intelligent matching criteria modification technique of FIG. 10. Starting point 1100 is configured with the maximum dimensionality and minimum granularity. A threshold significance criteria is determined in which a node must contain at least 30 patient descriptors, and N is configured to require at least four combinations of dimensionality and granularity having that threshold significance. During iteration 1, limbs 1110 and 1111 represent cases in which a dimension 1 and a dimension 2 have been reduced, respectively. Limb 1112 represents a limb in which the granularity of dimension 1 has been increased, and limb 1113 represents a case in which the granularity of dimension 2 has been increased. Each of the limbs within iteration 1 are evaluated for disparity in clustering patterns of records with negative outcomes from records with position outcomes. Limbs 1111 and 1113 are determined to have decreased disparity, and are therefore clipped. Limbs 1110 and 1112 are determined to have increased disparity, and are therefore preserved. Regardless of whether limbs 1110 and 1112 meet the threshold significance requirement, they number less than N, such that another iteration is conducted. In iteration 2, limb 1110 is branched to limb 1120 (further reducing dimension 2) and limb 1121 (increasing granularity of dimension 2). (It is assumed, for purposes of this example, that some additional dimension remains in limb 1120 even after reduction of dimensions 1 and 2.) Limb 1112 retains both dimensions 1 and 2, and is therefore further branched to evaluate four cases: subsequent reduction of dimension 1 (1122), subsequent reduction of dimension 2 (1123), increase in granularity of dimension 1 (1124) and increase in granularity of dimension 2 (1125). In iteration 2, limb 1120 is determined to have reduced disparity between clustering of negative and positive outcomes, and is therefore clipped. The remaining limbs 1121, 1122, 1123, 1124 and 1125 are determined to have positive disparity, and there therefore preserved. The preserved limbs are evaluated against the significance threshold and determined to exceed the threshold significance, such that the N limbs showing greatest disparity (1121, 1122, 1124 and 1125) are selected, such that their individual and aggregated predicted patient outcomes can be calculated.

### Barriers to Data Acquisition

While suitable data with which to perform many of the analyses described herein is available in hospitals of today, the information is often segregated into several disparate electronic storage systems with no easily-facilitated conduit to aggregate the information. There are various manners in which to delineate the types of information that may be desired to be fed into the platform for analysis. One manner of classifying the information types is to distribute them based on their source, e.g.: admission report, chemical laboratory results, radiation laboratory results, medical images, patient monitor data, clinician's notes, or manually recorded physiological measurements. A second manner of classifying the information types is to distribute them based on the manner by which they were recorded, e.g.: autonomously generated by EHR, manually entered by clinician, or autonomously recorded by physiological sensing device. Other ways of classifying data include: source (vital sign from monitor versus vital sign from EHR); extracted or not (e.g. a qualitative descriptor extracted from a waveform or via NLP, versus a non-extracted vital sign measurement); history or in-clinic (e.g. demographic information and prior medication information provided by a patient versus an in-clinic lab result or in hospital medication); free text data (e.g. processed via NLP) versus standardized text input (e.g. user input text in a structured field, not processed via NLP) versus automated data (e.g. lab result); signal quality passed from a previous measurement; and dynamic versus static (e.g. heart rate versus a lab result of something normally only tested once). These different means of information storage can serve as a framework for various methods of data acquisition.

In some embodiments, most of the information in a patient's admission report, as well as many of the patient's physiological measurements, are recorded into an Electronic Health Record (EHR) by a clinician, and stored in a standardized format. Such EHR systems may make patient information available via a data network. In some embodiments, information can be acquired from EHR 430A by server 400 for analysis via SQL query or HL7 messaging.

Some information may be stored in an EHR in non-standardized format. For example, nurse or clinician notes and qualitative information in the admission report may be stored in the EHR as free text data. In order to acquire and utilize information from free-text data in the EHR, some embodiments may implement natural language processing (NLP) techniques to identify and extract relevant information. In the embodiment of FIG. 6, application server 402 includes NLP component 622. EHR information obtained, e.g. in step 710 of the embodiment of FIG. 7 or step 800 of the embodiment of FIG. 8, is evaluated for identification of free text data. To the extent free text data is identified, it is then processed by NLP component 622 to extract concepts that may be utilized in the patient descriptor for analysis purposes.

Some information may be available for transfer via standardized formats. For example, lab and radiology information may be transferred via a predetermined protocol known as HL7. In some embodiments, the platform will access lab data on a particular patient as it becomes available through I/O scripts written to acquire information using the HL7 protocol. In some embodiments, such data may be acquired through HL7 via EHR 430A. In other embodiments, data may be acquired through HL7 via direct query to a network-accessible lab data system.

Some information may be acquired from patient monitors and physiologic sensing devices. Many patient monitors common in hospital environments are able to store a significant memory of patient measurements, but are not linked directly to the EHR. The platform may be augmented with I/O scripts that are calibrated to interface with various brands of patient monitors. Many patient monitors, particularly newer devices, may be accessed through software means, without the need for custom-built data extraction hardware. For older devices without appropriate software data interfaces, it may be desirable to provide data extraction hardware customized to retrieve data from the patient monitor or the physiological monitoring devices themselves and make it available via a software interface to the platform.

### Disease or Condition Specific Analyses

Some embodiments are described herein in the context of predicting outcome for a particular future condition, outcome or diagnosis (whether a binary determination, higher-level determination or continuous value output). However, it is contemplated and understood that the same devices, infrastructure, systems, methods and techniques can be readily employed in embodiments analyzing for a larger number of conditions, potential outcomes or diagnoses. For example, server 100 may utilize varying combinations of the patient physiological data available to it in order to simultaneously predict homeostatic instability, suggest a hospital ward for patient transfer, evaluate risk of sepsis and recommend a rate for application of fluids to the patient. Moreover, these analyses may be performed simultaneously for a large number of patients.

### Iterative Training and Evaluation

In some embodiments, as increasing numbers of patient descriptors are introduced into the system for purposes of evaluation, those descriptors may also be utilized to further train the system. In this way, the diagnostic effectiveness of the overall system may continually improve as the system is used. Also, in some circumstances it is possible that a medical service provider's patient population differs from the initial training population with respect to the likelihood of developing a particular condition, given a particular patient descriptor. By implementing an iterative training process with a particular provider's own patient data, the predictive capability of the system may continually be optimized for that medical service provider's patient population.

FIG. 1B is an embodiment of a process through which the training system of FIG. 2 may interact with the evaluation system of FIG. 4 to provide iterative training. Analogously to FIG. 1A, in step 150 an initial set of training data is received by training server 200. In step 155, the system is trained using that data, as described elsewhere herein. In step 160, the results of the training exercise are installed into the evaluation environment; e.g. contents from trajectory lookup table probabilities 252, training data library 254 and calibrations constants 256 are copied into trajectory lookup table 636, patient data library 632 and calibration constant data store 634, respectively. In step 165, evaluation server 400 is utilized, such as via the process of FIG. 7 or 8, in order to evaluate the conditions of various patients given their recorded patient descriptors.

In step 170, patient descriptor data from patients evaluated in step 165, are fed back into the training data library. For example, new patient data recorded within data store 632 may be copied into training data repository 254. Then, training process 155 is repeated, but incorporating the patient data added in step 170 into the analysis. New classification mechanisms are determined in step 155 in view of the supplemented data set, installed back into the evaluation environment in step 160, and utilized for further evaluations in step 165.

The process of FIG. 1B illustrates a batch update embodiment in which feedback of patient data into the training library happens periodically. For example, the copying operation of step 170 may take place in connection with a periodic data warehousing process in which the patient data is also being archived (e.g. once every 6 weeks or once every 3 months). In other embodiments, the supplementation of the training library with new patient data may take place more regularly, potentially via automated network copying over a secure hospital network.

In yet other embodiments, patient data may be imported into the training library more frequently, such as daily, or even nearly immediately upon evaluation. The training process could be conducted immediately with each update, or it could be conducted at less frequent intervals that the intervals at which library updates take place. FIG. 1C illustrates a variation of the process of FIG. 1B. After new patient data is copied into the training library in step 170, a determination is made as to whether retraining should be conducted (step 175). If so, operation continues to training step 155. If not, the system continues executing additional patient evaluations (step 165). Thus, the frequency of training library updates and the frequency of training events are decoupled and can be determined independently.

### Utilization of Time-Series Patient Descriptors

In some embodiments, patient descriptors may include time series data. Patient vital signs are often measured periodically over time and stored within a patient's electronic health record and/or within patient monitoring equipment. A patient's physiological parameters can be trended over time in order to obtain additional insight into a patient's current or projected future condition.

One challenge in implementing a decision support system utilizing time series data is managing the time scale of patient descriptor data. The timing of various physiological measurements is generally not synchronized across patient monitoring devices. Some devices may take measurements at a greater frequency than others. Even instruments configured to take measurements at the same frequency may be offset relative to one another. Time also introduces increased dimensionality in patient descriptor data.

Some of these time series challenges are addressed by U.S. Patent Application No. 2008/0281170A1 (“the '170 application”). The '170 application proposes to normalize the time axis of time series data in order to achieve consistency in time scale between different time series parameters. For example, if temperature is measured continuously, blood pressure measured hourly, and white blood cell count measured daily, the '170 application proposes to normalize those parameters to match the time scale of the least frequent measurement. E.g. averaging temperature over the course of each hour to use with hourly blood pressure measurements in an analysis. Or averaging temperature and blood pressure over the course of a day to use with daily white blood cell counts in an analysis.

Different approaches to managing time series data streams may provide high levels of control over data complexity, while also enabling dynamic control over the tradeoff between temporal content and the statistical significance of each data point within the library data and current patient descriptor. As the time resolution of data utilized becomes finer, it becomes increasingly less likely that any patient descriptors in the library data will match the patient descriptor under analysis. Therefore, it may be desirable to utilize one or more of several techniques in order to implement embodiments utilizing time series data.

In some embodiments, it may be desirable to implement category-based time-difference incorporation. This principal enables different treatment of time differences based on the nature of the physiological parameter at issue. Amongst the categories that may be used as a basis for category-based time-difference incorporation are those described hereinabove. For some vital signs, the time difference between two different vital signs may be ignored, as standard practice calls for different monitoring frequencies and the differences are diagnostically insignificant. For other vital signs, the differences are diagnostically important and analytically accommodated, such as via normalization or rate of change calculation. Time differences may be particularly critical for lab measurements. For example, in cardiac lab testing, it may be inadequate to only know the difference between two different troponin measures without knowing the difference in time elapsed between the administrations of the two troponin tests. In some circumstances, the rate of change in a physiological parameter may be similarly or even more diagnostically significant than the absolute measures. By utilizing diagnostic categories to selectively ignore or account for time differences (such as via normalization or rate of change calculation), diagnostic capability may be maximized without unnecessary computational overhead.

Another mechanism that may be implemented in connection with patient descriptors having time series data is a “series of bricks” analysis. With the series of bricks approach, patient descriptors are mapped into a FDHS which is divided into a series of regions. If a patient's trajectory passes through a set of regions in a particular order, as informed by supervised machine learning analysis of prior patient trajectories, the system can assign some significance to that, such as correlating the trajectory with an anticipated outcome or condition. Dividing the FDHS into regions and mapping patient descriptors into those regions enables optimization of the tradeoff between getting as unique as possible a description of each patient, and enabling other patients in the library to have the same description.

A simple example of a series of bricks analysis that facilitates visualization is defining the finite discrete space as a three dimensional space, where the value of a different physiological measurement is mapped onto each dimension. At a single point in time, the value of the three measurements defines a point within the finite discrete space. If periodically, each of the three measurements are taken, those points can be plotted within the three dimensional space to yield a series of dots. If the dots are connected, the result is a time-parameterized trajectory through the three dimensional space. However, the time-parameterized trajectory of a single patient will seldom be exactly identical to the trajectory of any other patient. Therefore, to facilitate common classification of trajectories, a binning method can be utilized. In the exemplary three dimensional space, binning is analogous to chopping the space into a regular pile of bricks, e.g. cuboids. The binned time parameterized trajectory now looks like a series of bricks that are lit up in a particular order. One advantage of this binning process is that it increases the statistical significance of any particular “series of bricks” trajectory. Another advantage of the binning process is that it reduces the computational intensity of the algorithm. One possible series of bricks trajectory is a single brick being lit up over several sample points; this may indicate a homeostatic stable condition. Another possible series of bricks trajectory is two bricks between which the trajectory oscillates back and forth; this may indicate an oscillatory condition. Another series of bricks trajectory is a series of unique bricks through which the trajectory travels; this may indicate a condition that is undergoing a shift, potentially becoming more stable or more unstable. In any case, a library of “series of bricks trajectories” can be built up on different time scales or sampling rates, each trajectory associated with a particular prior outcome. Some of the “series of bricks” trajectories may be associated with, e.g., a more stable outcome than others, and this correlation can be used when computing a final score for a patient, or otherwise providing a final condition evaluation.

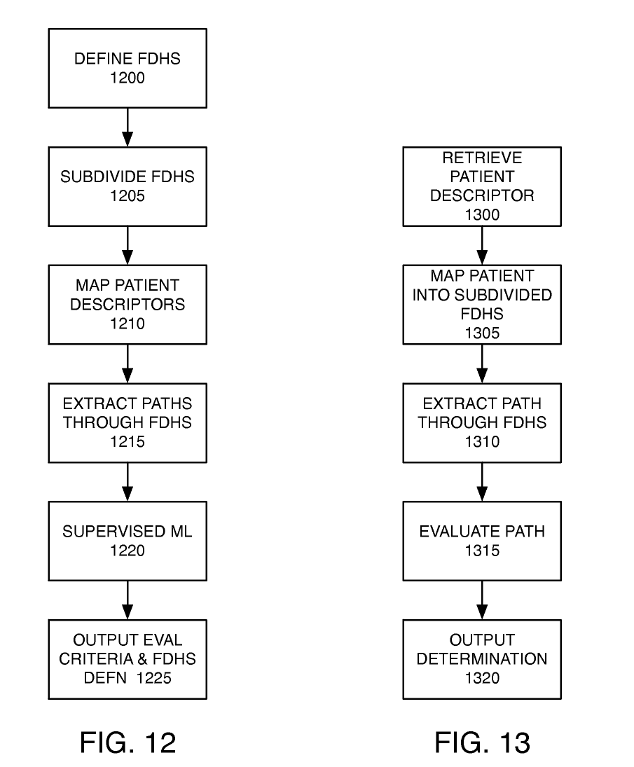

FIG. 12 illustrates such a mechanism. In a training process, in step 1200, a finite discrete hyperdimensional space is defined. In step 1205, the FDHS is subdivided into regions defined by ranges within each dimension (thus creating the conceptual “bricks”). In step 1210, time series patient descriptors from a library of prior patient data and actual outcomes, are mapped into the FDHS. In step 1215, the path of each patient descriptor through the subdivided FDHS is extracted. In step 1220, the paths extracted in step 1215 are utilized as inputs to a supervised machine learning process, along with the corresponding outcome associated with the patient descriptor from which each path was derived. In some embodiments, the output of step 1215 may be absolute paths, i.e. a sequence of specific regions through which a trajectory passes. In other embodiments, the output of step 1215 may be relative paths, i.e. delta steps relative to a starting point. In yet other embodiments, some combination of absolute positioning within a FDHS along with relative trajectory may be utilized. In step 1225, the resulting optimized evaluation criteria (which may be linear, nonlinear, or some combination of linear and nonlinear elements, as best for a particular analysis) and subdivided FDHS definition are output, for subsequent use in the clinical environment.

FIG. 13 illustrates a process for using the results of FIG. 12 in the clinical environment. In step 1300, a patient descriptor having time-series data is retrieved from an EHR and/or other patient monitoring resources, as described in connection with other embodiments above. In step 1305, the current patient descriptor is mapped into the subdivided FDHS as defined in the output of step 1225. In step 1310, the patient's path through the subdivided FDHS is extracted. In step 1315, the extracted path is evaluated against the algorithm or criteria identified in step 1225, towards determining a condition determination or projected outcome. In step 1320, the output determination is returned, e.g. to a requesting clinician or for storage and processing by another data system.

The way in which the FDHS is subdivided may be an important factor in the effectiveness of any particular evaluation. If the FDHS is subdivided too finely, or if a training library is relatively limited, the system may be unlikely to identify prior patients in the library having the same trajectory. Conversely, if the FDHS is subdivided too coarsely, a strong correlation between bricked trajectory and clinical outcome may be sacrificed. Also, if a relatively large training library is available, statistically valuable results may still be obtained even with comparatively fine subdivision of the FDHS. Also, in some applications it may be determined that different coarseness or granularity levels may be applied to different measurement axes in order to optimize results.

Several approaches to optimizing subdivision of the FDHS may be utilized. In some embodiments, the FDHS may be subdivided based on fixed granularity in a fixed number of dimensions. Clinical literature may be utilized to guide identification of appropriate dimensionality and granularity for any given type of evaluation being performed. An alternative approach is dynamic FDHS subdivision. Dynamic FDHS subdivision is analogous to the dynamic multiparameter calibration techniques described above, e.g. in connection with FIGS. 9-11.

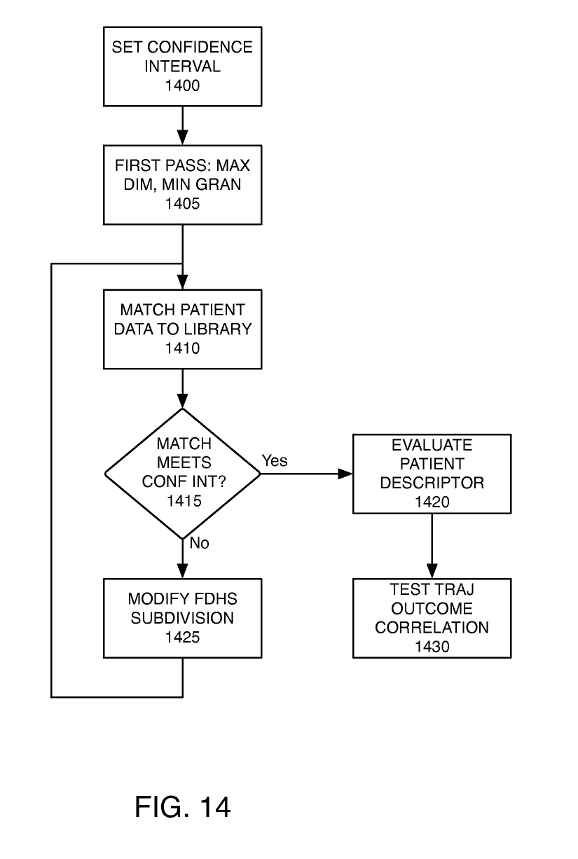

FIG. 14 illustrates one embodiment of a mechanism for dynamic FDHS subdivision. In step 1400, a target confidence interval is defined, e.g. a minimum number of prior patient descriptor trajectories that match the current patient trajectory. In step 1405, an initial subdivision is applied to a FDHS into which library patient descriptors and the current patient descriptor are mapped. In step 1410, server 400 evaluates the number of library patient descriptor trajectories that match the current patient descriptor trajectory. In step 1415, a determination is made by server 400 as to whether the confidence interval of step 1400 is satisfied, e.g. whether the number of library patient descriptors having trajectories matching the current patient descriptor trajectory exceeds a threshold number. If so, the current patient is evaluated using a series of bricks evaluation technique with the current FDHS subdivision (step 1420). If not, the FDHS subdivision is modified (step 1425), and the process returns to step 1410 for another iteration. The modification in step 1425 typically involves increasing the granularity along one or more dimensions. In some embodiments, the modification in step 1425 may also involve dimensional reduction.

Eventually, the FDHS subdivision and dimensionality is such that the confidence interval test of step 1415 is met, and the patient descriptor is evaluated in step 1420. In some embodiments, it may be desirable to further evaluate the result quality. If there are few library trajectories similar to the current patient trajectory, it is possible that FDHS granularity and dimensionality is reduced to a point where the final trajectory no longer exhibits a high level of correlation between trajectory and projected outcome. Therefore, in step 1430, the correlation of library trajectories matching the current trajectory to library outcomes is tested, and optionally reported along with the result.

Another important factor in implementing the mechanism of FIG. 14 is determining how to modify the FDHS subdivision. For example, server 400 must determine which one or more dimensions in the FDHS will be modified, and the degree to which the granularity should be adjusted for each dimension that is modified. Multiple different ways of subdividing the FDHS may achieve the desired confidence interval. Therefore, in some embodiments, it may be desirable to implement multiple instances of the mechanism of FIG. 14, each with different approaches to modifying dimensionality and/or granularity of the FDHS, and each defining the “series of bricks” corresponding to the current patient's trajectory through the FDHS in a different way. Then, whichever result yields the highest correlation between trajectory and outcome based on library data is selected for evaluation of the result for the current patient trajectory.

In some embodiments of a dynamic FDHS subdivision technique, it may also be desirable to also dynamically vary the time scale, e.g. by modifying the time parameterization of the trajectories. Multi-time scale monitoring processes analogous to those described further below can be incorporated into a dynamic FDHS subdivision mechanism.

Another characteristic of some patient evaluations using time-series data is that more recent physiological measurements may have a higher correlation to the patient's condition or projected outcome than older measurement. Different techniques can be utilized to account for this factor in the analysis mechanism. In some embodiments, the length of patient trajectory subject to examination may be limited. For example, if it is believed that a particular condition being evaluated develops and exhibits itself within a period of 48 hours, the analyses described herein may be applied only to physiological measurements taken within a 48 hour period. In other words, the trajectory length within the FDHS is capped at 48 hours (typically the most recent 48 hours from the time of evaluation).

An alternative approach is to apply time-based weighting of physiological data in the patient descriptor within a scoring or evaluation mechanism. While older measurements may still exhibit some level of correlation, more recent measurements may be more indicative of a patient's current and upcoming state than older measurements. In such embodiments, an analysis may apply a descending weight to measurements that are further back in time from the time of the most recent data.

In some embodiments, time series physiological data may be available that describes a waveform. It may be desirable to pre-process such waveform data in order to yield information in a form more useful to the training and classification mechanisms described elsewhere herein. To that end, application server 402 may include waveform preprocessing component 624. In some embodiments, waveform preprocessing component 624 may apply a noise reduction process to patient waveform data prior to analysis of the data for training or evaluation purposes. In some embodiments, waveform preprocessing component 624 may extract one or more features from waveform data, i.e. extracting non-continuous information out of a continuous waveform. The extracted feature(s) may then be utilized as analysis inputs rather than the raw waveform data itself. Examples of potential extracted features include, inter alia, the signal quality of the waveform (i.e. lack of noise, artifacts, or coupling of 60 Hz/powerline interference), the pulse amplitude of the waveform, the pulse frequency of the waveform, and the normality of the waveform versus expected forms (i.e. does the ECG qualitatively look like a healthy ECG?).

On training server 200, an analogous waveform preprocessing component (not shown) may be implemented as a subcomponent within acquire and process component 220 in order to process waveform data within the library patient descriptors for use in a training process.

### Multi-Time Scale Monitoring

The prior art '170 application addresses normalization of the time axis of time series data in order to achieve consistency in time scale between different time series parameters. The examples described involve normalizing higher-frequency measurements to match the time scale of lower-frequency measurements.

In accordance with another aspect of the systems and methods described herein, a dynamic time scale monitoring mechanism is provided. This variation on the dynamic FDHS approach described above dynamically selects a time scale to not only facilitate computation, but also optimize for output quality. More specifically, multiple different time scales can be applied to time series data, potentially revealing patterns in one time scale that are masked in others. For example, if the patient descriptor includes multiple vital signs on different time scales and the least frequent sampling rate is 1 hour, any measurement frequency over 1 hour is unnecessary for multi-measurement compatibility. However, it may still be desirable to monitor vital signs on a more infrequent rate (e.g. 2 hours, 4 hours, daily, etc.) to identify trends that manifest on different time scales.

Alternatively, in some embodiments it may be desirable to utilize time scales having intervals shorter than the sampling period of the measurement having the lowest available sampling rate. In such an embodiment, one approach to handling measurements with lower sample rates is to repeat the last-recorded value of a “slow measurement” until a new value becomes available. This technique is often effective in typical clinical environments, as measurements taken less frequently are typically more stable by nature, or they typically change more slowly, or are considered of less concern to the clinician than rapidly sampled measurements. Thus, the patient is assumed to have the most recently recorded value of each measurement, unless/until a new value is taken. In addition to enabling multi time scale processing of patient data streams with different sample rates, this mechanism also allows for graceful handling of missed data.

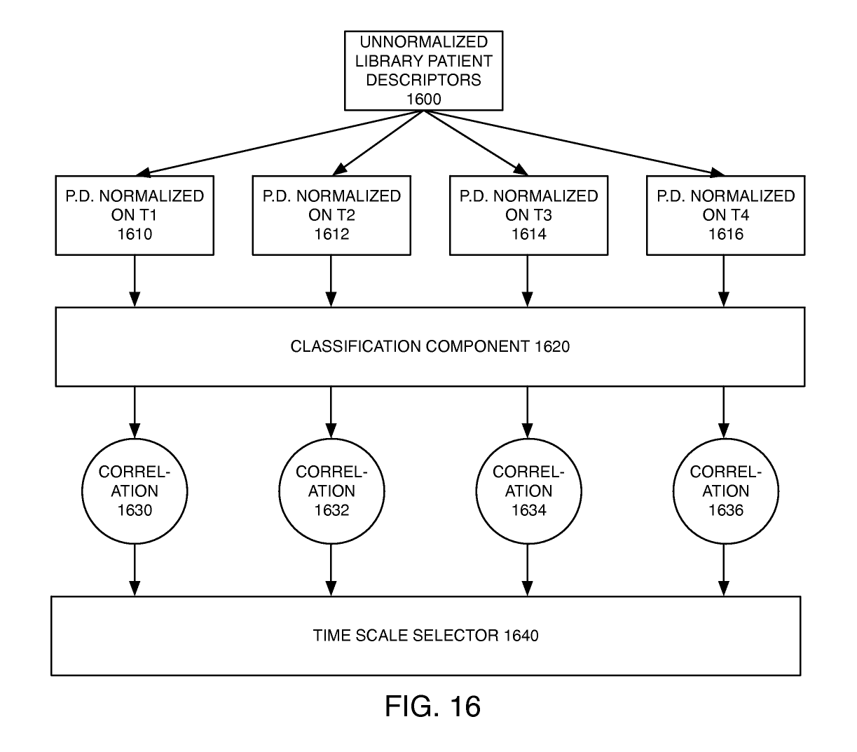

FIG. 16 illustrates one embodiment of a mechanism for implementing multi-time scale monitoring in connection with a training process. Unnormalized patient descriptors library 1600 is fed to time scale normalization components 1610, 1612, 1614 and 1616, each of which normalized patient descriptor data 1600 on different time scales T1, T2, T3 and T4, respectively. The differently-normalized patient descriptor data is then fed to classification component 1620, which may be a supervised machine learning mechanism as described elsewhere herein. The classification results and trained algorithm coefficients for data normalized on each of time scales T1, T2, T3 and T4 are fed to correlation evaluation components 1630, 1632, 1634 and 1636, respectively, each of which evaluates the accuracy of trained algorithm. Based on the output of evaluation components 1630, 1632, 1634 and 1636, time scale selector 1640 identifies the optimal time scale or time scales from amongst T1, T2, T3 and T4.

### Differentiation Between Endogenous and Exogenous Events

Another factor that may be important in evaluating a patient descriptor is differentiation between endogenous and exogenous effects. Exogenous effects are physiologic changes brought about through an act that is external to the patient including, but not limited to, a particular treatment the patient has undergone or a particular medication that the patient is taking (e.g. drug administration, fluid delivery, cardiac resuscitation, or surgery). Endogenous effects are physiologic changes brought about through mechanisms internal to the patient, typically as a result of the homeostatic mechanisms within the patient. Prior systems are often blind to differences between exogenous and endogenous changes. This is problematic because a decision support system may erroneously determine, for example, that a patient is stable, when the patient is in fact unstable but being supported artificially. Alternatively, a stability assessment may determine that a patient is unstable when the patient exhibits symptoms that are natural, and safe results of therapies applied to them.

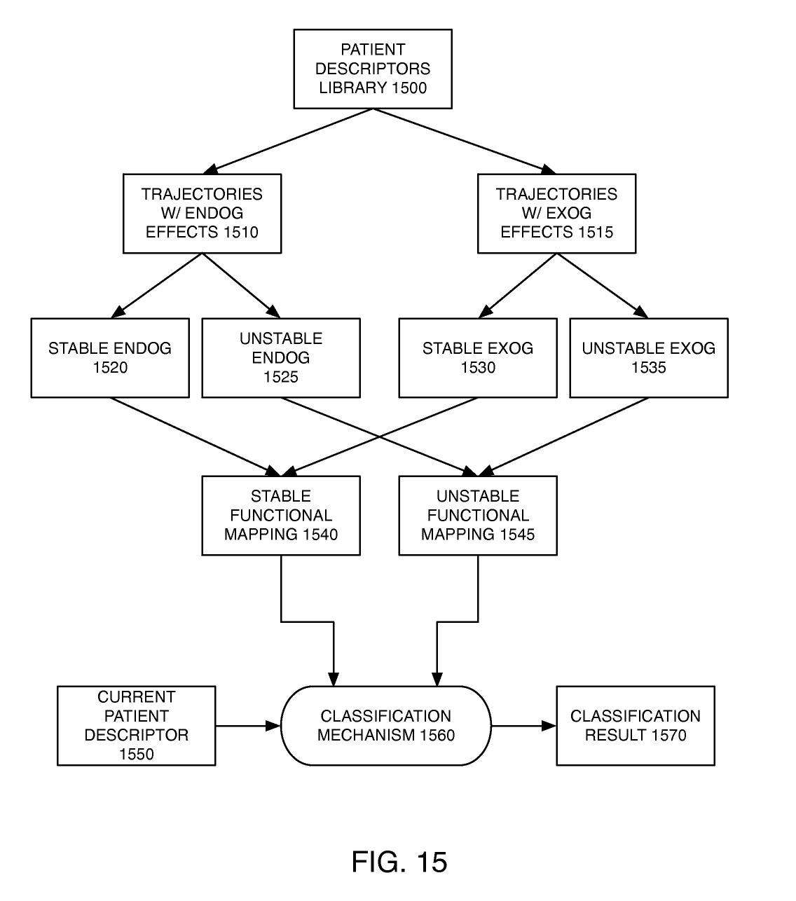

In some embodiments, the FDHS space and trajectory analysis techniques described herein are implemented in a manner that discriminates between exogenous and endogenous changes. In accordance with one such embodiment, illustrated in FIG. 15, a training library of patient descriptors 1500 includes identification of exogenous changes (e.g. clinical interventions) applicable to each patient. Such clinical interventions may be classified within the patient descriptors as quantitative interpretive data, as described elsewhere herein. For each of these types of interventions a time window will be assigned, within which significant changes in the patient's state in a FDHS will be attributed to the exogenous effect. However, if no clinical intervention occurred and a significant shift occurs in the FDHS, the change is attributed to an endogenous effect. The goal is to identify unique patterns in the trajectory shift that allow for statistically accurate discrimination between endogenous shifts and exogenous shifts. Thus, when a classification mechanism is subsequently used to evaluate a patient, the mechanism will be able to identify an exogenous shift, even if the particular clinical intervention that caused the shift has not been recorded. This therefore allows the mechanism to appropriately interpret the shift.

In the embodiment of FIG. 15, training library 1500 is divided into a repository 1510 of all trajectories that are statistically correlated with endogenous changes, and a repository 1515 of all trajectories that are statistically correlated with exogenous changes, each compiled through use of supervised machine learning techniques on retrospective patient datasets. In a stability assessment, for example, four trajectory repositories are created: stable with endogenous changes (repository 1520), stable with exogenous changes (repository 1530), unstable with endogenous changes (repository 1525) and unstable with exogenous changes (repository 1535). Functional mappings 1540 and 1545 are formed that each compensate for exogenous changes, similarly to adjustments for quantitative interpretation parameters, so that they do not manifest as erroneous artifacts in the final stability assessment. For example, predetermined thresholds within the analysis may be readjusted. During evaluation of a current patient descriptor, a determination as to whether a current patient descriptor is subject to exogenous effects can be determined via, e.g., real-time NLP of the current patient descriptor, or coded EHR data lookup. Exogenous impact mappings 1540 and 1545 can then be applied to classification mechanism 1560 (which may include classification mechanisms described elsewhere herein) in order to generate classification result 1570 based on current patient descriptor 1550.

While certain embodiments of the invention have been described herein in detail for purposes of clarity and understanding, the foregoing description and Figures merely explain and illustrate the present invention and the present invention is not limited thereto. It will be appreciated that those skilled in the art, having the present disclosure before them, will be able to make modifications and variations to that disclosed herein without departing from the scope of the invention or appended claims.

1. A system for evaluating a condition of an individual through analysis of physiological data associated with the individual, the system comprising:
a data collection component that receives a patient descriptor from patient monitoring equipment, the patient descriptor comprising time series physiological values associated with the individual, the time series physiological values forming a trajectory within a finite discrete multidimensional space (FDMS); and
a data analysis component implemented by an analysis server, the data analysis component applying a classification component to the patient descriptor to yield a condition of the individual, the classification component mapping the patient descriptor into the FDMS, and applying weighting factors to each physiological value, the weighting factors varying at least in part based on a time associated with each physiological value.

2. The system of claim 1, in which the weighting factors are configured to apply varying significance to points within each trajectory at varying points in time.

3. The system of claim 2, in which one or more of the weighting factors further varies based on the frequency with which an associated type of measurement is recorded within the patient descriptor.

4. The system of claim 3, in which said one or more of the weighting factors increases with the frequency with which an associated type of measurement is recorded within the patient descriptor.

5. A method for evaluating a condition of an individual through analysis of physiological data associated with the individual, the method comprising:
receiving a set of training data, the training data comprising a plurality of library patient descriptors, each of said library patient descriptors associated with a training data outcome, and where each of the library patient descriptors comprises time-series physiological data;
deriving a classification mechanism and time scale normalization mechanism for evaluating a condition of the individual by:
generating versions of the library patient descriptors, each utilizing a different version of a time scale normalization mechanism;
for each library patient descriptors version, applying a computational optimization component to generate a classification mechanism option;
evaluating the classification mechanism options to select a classification mechanism and corresponding time scale normalization mechanism; and
applying the time scale normalization mechanism and classification mechanism to the physiological data associated with the individual to evaluate the condition of the individual.

6. The method of claim 5, in which the computational optimization component comprises a supervised machine learning component.

7. The method of claim 5, in which the step of evaluating the classification mechanism options comprises selecting a subset of the classification mechanism options having the greatest correlation between predicted outcomes and training data outcomes for the library patient descriptors.

8. The method of claim 5, in which each time scale normalization mechanism normalizes time series information associated with data within the library patient descriptors on a different time scale.

9. The method of claim 5, in which the time scale normalization mechanism normalizes physiological data within each library patient descriptor on one of a plurality of different time scales, based upon the association of one of a plurality of categories with the physiological data.

10. The method of claim 9, in which the plurality of categories comprises: dynamic measurements and static measurements.

11. The method of claim 5, in which the time scale normalization mechanism normalizes a first subset of time series data within each library patient descriptor, and refrains from normalizing a second subset of time series data within each library patient descriptor.

12. A method for evaluating a condition of an individual from amongst a plurality of possible conditions through analysis of physiological data associated with the individual, the method comprising:
receiving a set of training data, the training data comprising a plurality of library patient descriptors comprising physiological data and data indicative of the presence or absence of an exogenous event, each of the library patient descriptors associated with a training data outcome;
deriving a functional mapping associated with each of the plurality of possible conditions, each mapping normalizing library patient descriptors to compensate for the exogenous event, if present;
applying the functional mappings to a subject patient descriptor comprising the physiological data associated with the individual to generate a compensated subject patient descriptor; and
applying a classification mechanism to the compensated subject patient descriptor to evaluate the condition of the individual.

13. The method of claim 12, in which the step of applying the functional mappings to a subject patient descriptor comprises processing the subject patient descriptor using a natural language processing component to determine the presence or absence of the exogenous event.

14. The method of claim 13, in which the step of applying the functional mappings to a subject patient descriptor comprises performing a data query of an electronic health record system or bedside patient monitor to determine the presence or absence of the exogenous event.

15. The method of claim 14, in which the step of performing a data query of an electronic health record to determine the presence or absence of the exogenous event comprises determining whether blood pressure medication as been applied to the individual.

16. The method of claim 12 in which the training data comprises time series data, and the subject patient descriptor comprises time series data.
